(19) United States
(12) Patent Application Publication
(10) Pub. No.: US 2021/0300863 A1
(43) Pub. Date: Sep. 30, 2021

(54) PROCESS TO MAKE A SELECTIVE CATHEPSIN CYSTEINE PROTEASE INHIBITOR

(71) Applicant: Intervet Inc., Madison, NJ (US)

(72) Inventors: Eric Voight, Pleasant Prairie, WI (US); Christophe Pierre Alain Chassaing, Ingelheim am Rhein (DE); Hans Peter Niedermann, Bubenheim (DE); Stephan Veit, Budenheim (DE); Claudia Scheipers, Ingelheim (DE)

(73) Assignee: Intervet Inc., Madison, NJ (US)

(21) Appl. No.: 17/264,394

(22) PCT Filed: Aug. 1, 2019
(86) PCT No.: PCT/EP2019/070766

§ 371 Date: Jan. 29, 2021

(30) Foreign Application Priority Data
Aug. 2, 2018 (EP) 18187046.0

(51) Int. Cl. C07C 253/30

(52) U.S. Cl. CPC C07C 253/30; C07C 2601/16; C07C 2601/02

(57) ABSTRACT

A method of preparing a compound of Formula (I) comprising reacting the compound of Formula (A) with a base and a compound of Formula (B) to yield a compound of Formula (C).

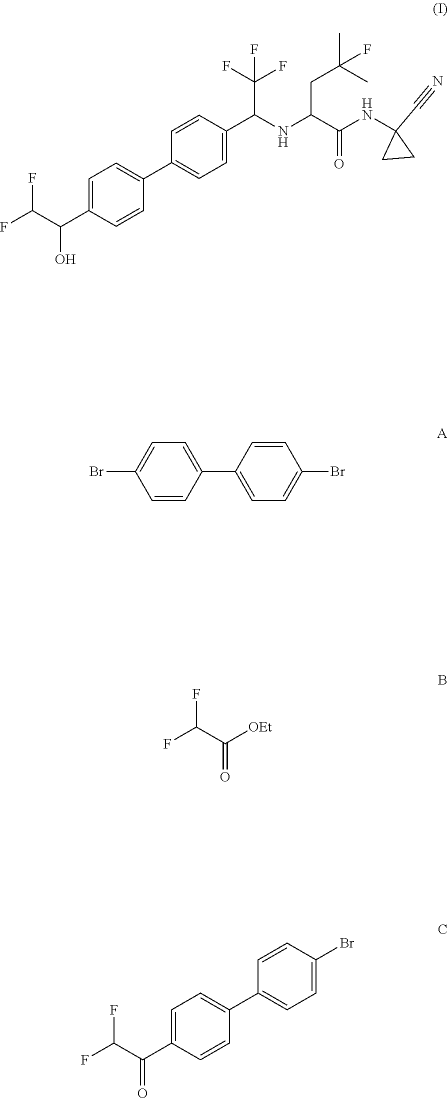 [SMILES: CC(C)(F)CC(NC(c1ccc(-c2ccc(C(O)C(F)F)cc2)cc1)C(F)(F)F)C(=O)NC1(C#N)CC1 ; Brc1ccc(-c2ccc(Br)cc2)cc1 ; CCOC(=O)C(F)F ; O=C(c1ccc(-c2ccc(Br)cc2)cc1)C(F)F]

### BACKGROUND

U.S. Pat. No. 7,407,959 discloses compounds of the following formula which are selective cathepsin cysteine protease inhibitors and can thus be used in the treatment of diseases such as osteoarthritis or osteoporosis. Processes to make these compounds are also disclosed.

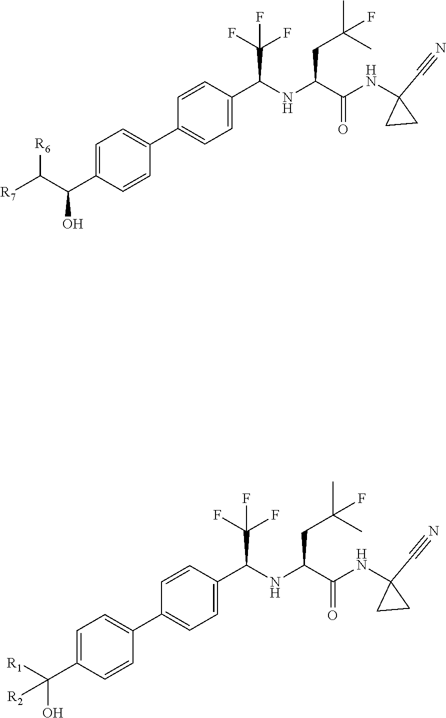

WO2006/017455 discloses processes for diastereoselective reductive amination whereby perfluorinated ketones or ketals are reacted with α-aminoesters to form imine metal carboxylates which are stereoselectively reduced. Specifically, the synthesis of 4-fluor-N-{(1S)-2,2,2-trifluoro-1-[4′-(methylsulfonyl)biphenyl-4-yl]ethyl}-L-leucine dicyclohexylamine salt is disclosed (see pp 18-19):

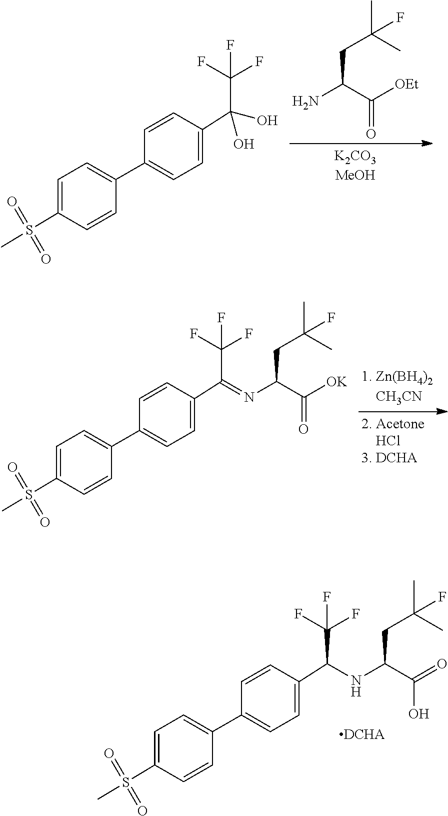

WO2012/148555 discloses an amidation process whereby perfluorinated amino acids incorporating either a [4-(4-methylsulfonylphenyl)phenyl] or a [4-(4-methylsulfinylphenyl)phenyl] group, such as those described in the previous citation, can be treated with an amine in the presence of a coupling agent to yield the corresponding amides. Specifically, the synthesis of N-(1-cyanocyclopropyl)-4-fluoro-N²-{(1S)-2,2,2-trifluoro-1-[4′-(methylsulfonyl)biphenyl-4-yl]ethyl}-L-leucinamide is disclosed (see pp 8-9):

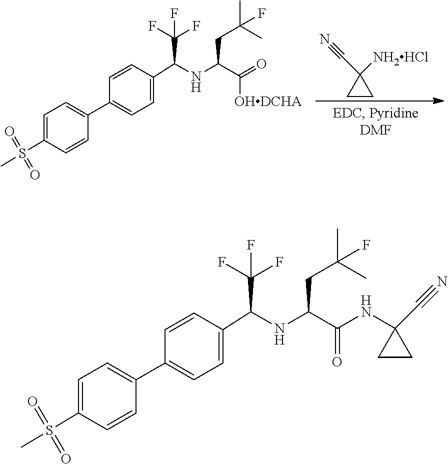

Isabel et al., Bioorg. Med. Chem. Lett. 2010, 20(3), 887-892, discloses the discovery of MK-0674, an orally bioavailable cathepsin K inhibitor. Unlike described in the previously cited references, the synthesis of the compounds is articulated around a key Suzuki coupling step between a highly functionalized boronic ester and an aryl bromide or chloride:

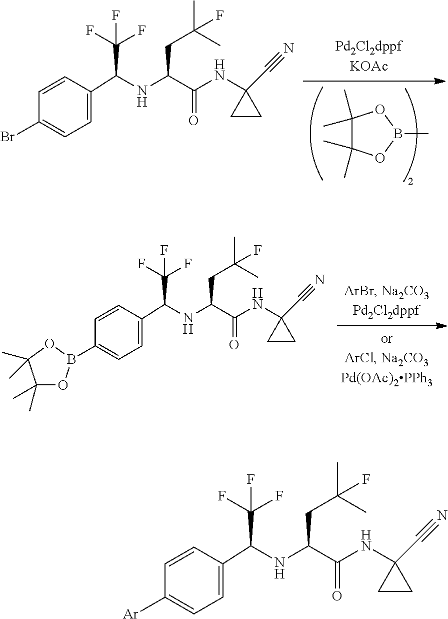

wherein Ar includes inter alia

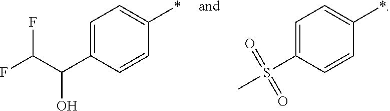

The synthesis pathway towards the final product involves seven steps and the repeated use of expensive palladium catalysts.

Following a similar strategy but inverting the reactive groups of the Suzuki coupling partners, O'Shea, et al., J. Org. Chem. 2009, 74(4), 1605-1610, discloses an enantioselective synthesis of odanacatib via the Suzuki coupling of a highly functionalized bromide with an arylboronic ester; the preparation of the functionalized bromide being achieved via a stereoselective nucleophilic displacement of an α-trifluoromethylbenzyl triflate.

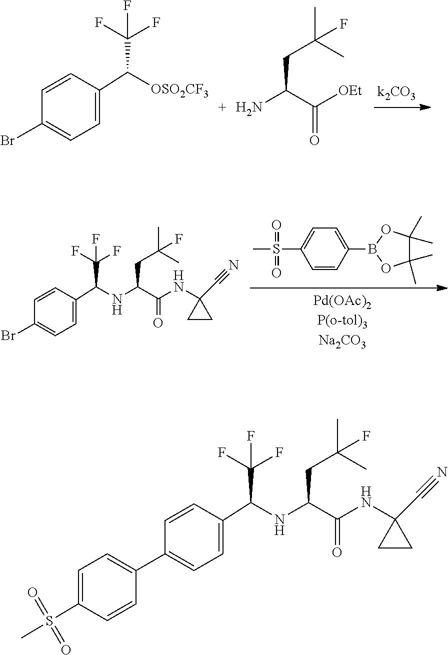

WO2006/133559 discloses (1R)-1-(4′-bromobiphenyl-4-yl)-2,2-difluoroethanol and 1-{4′-[(1R)-2,2-1-hydroxylethyl]biphenyl-4-yl}-2,2,2-trifluoroethanone (see Examples 65 and 66 respectively) which may be required for performing part of the chemistry disclosed in previously mentioned citations.

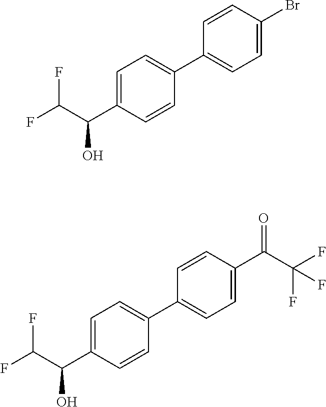 [SMILES: O[C@H](c1ccc(-c2ccc(Br)cc2)cc1)C(F)F ; O=C(c1ccc(-c2ccc([C@@H](O)C(F)F)cc2)cc1)C(F)(F)F]

Dolman, et al., Tetrahedron 2006, 62, 5092-5098, discloses a selective metal-halogen exchange of 4,4′-dibromobiphenyl mediated by lithium tributylmagnesiate.

Fujisawa, et al., Tetrahedron 1998, 54, 4267-4276, discloses the preparation of 4-bromo-4′-(trifluoroacetyl)biphenyl by trifluoracetylation of 4,4′-dibromobiphenyl.

WO2013/148554 discloses a process for the preparation and the chiral resolution of γ-fluoroleucine alkyl esters which are also key components of cathepsin cysteine protease inhibitors such as those disclosed in U.S. Pat. No. 7,407,959. Specifically, the synthesis of the following compounds is disclosed:

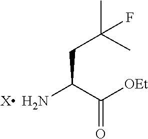

wherein X is H₂SO₄, L-tartaric acid, D-BOC proline, D-(+)-10-camphorsulfonic acid or N-acetyl-D-phenyl alanine.

N¹-(1-cyanocycloproply)-N²-((1S)-1-{4′-[(1R-2,2-difluoro-1-hydroxyethyl]biphenyl-4-yl}-2,2,2-trifluoroethyl)-4-fluoro-L-leucinamide (MK-0674) is orally bioavailable cathepsin K inhibitor compound having the structure of Formula Ia.

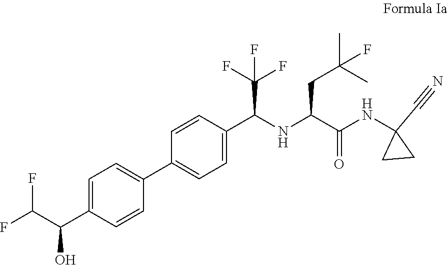 [SMILES: CC(C)(F)C[C@H](N[C@@H](c1ccc(-c2ccc([C@@H](O)C(F)F)cc2)cc1)C(F)(F)F)C(=O)NC1(C#N)CC1]

The IUPAC name of this compound is (2S)-N-(1-cyanocyclopropyl)-2-[[(1S)-1-[4-[4-[(1R)-2,2-difluoro-1-hydroxy-ethyl]phenyl]phenyl]-2,2,2-trifluoro-ethyl]amino]-4-fluoro-4-methyl-pentanamide.

Prior art syntheses of this compound have focused on functionalizing each of the phenyl ring separately and on coupling the resulting highly substituted aromatic intermediates to form the biphenyl core (See scheme 1 below)

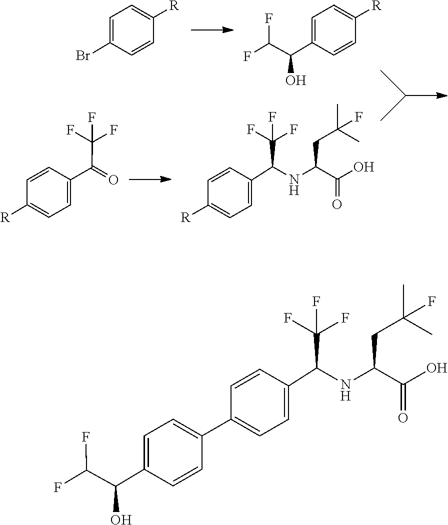

### SUMMARY OF THE INVENTION

Applicants have developed a more advantageous process in which the biphenyl structure is already present in the starting material. Compared to known methods, this process has fewer synthetic steps, high overall yield and does not need expensive and eventually toxic organometallic catalysts. (See scheme 2 below.)

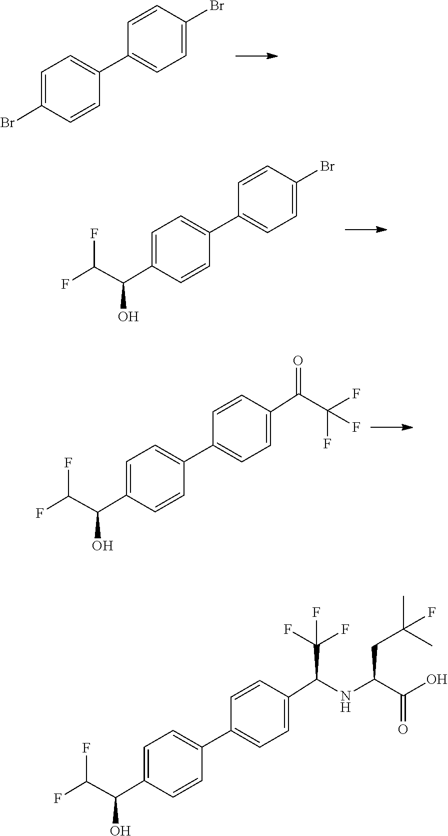 [SMILES: Brc1ccc(-c2ccc(Br)cc2)cc1 ; O[C@H](c1ccc(-c2ccc(Br)cc2)cc1)C(F)F ; O=C(c1ccc(-c2ccc([C@@H](O)C(F)F)cc2)cc1)C(F)(F)F ; CC(C)(F)C[C@H](N[C@@H](c1ccc(-c2ccc([C@@H](O)C(F)F)cc2)cc1)C(F)(F)F)C(=O)O]

Applicants have also found that temperature control on the reaction sequence can impact the yield and optical purity of the reaction scheme.

An embodiment of the invention is a method of preparing a compound of Formula I

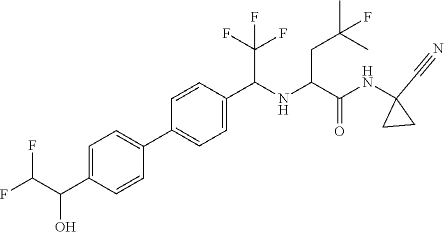 [SMILES: CC(C)(F)CC(NC(c1ccc(-c2ccc(C(O)C(F)F)cc2)cc1)C(F)(F)F)C(=O)NC1(C#N)CC1]

comprising
reacting the compound of Formula A

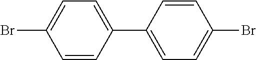 [SMILES: Brc1ccc(-c2ccc(Br)cc2)cc1]

with a base and a compound of Formula B

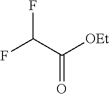

to yield a compound of Formula C

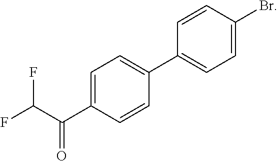 [SMILES: O=C(c1ccc(-c2ccc(Br)cc2)cc1)C(F)F]

### DETAILED DESCRIPTION

### Definitions

Dean-Stark apparatus refers to a configuration of laboratory glassware used to collect water or other liquid from a reactor.

THF means tetrahydrofuran.

TMEDA means N,N,N′,N′-tetramethylethane-1,2-diamine.

MTBE is methyl tert-butyl ether.

NMM is N-methylmorpholine also called 4-methylmorpholine.

HATU is O-(7-azobenzotriazol-1-yl)-1,1,3,3-tetramethyluronium hexafluorophosphate also known as 1-[Bis(dimethylamino)methylene]-1H-1,2,3-triazolo[4,5-b]pyridinium 3-oxid hexafluorophosphate or Hexafluorophosphate Azabenzotriazole Tetramethyl Uronium.

DMF refers to N,N-dimethylformamide.

Alkyllithium compounds and organolithium reagents are reagents that contain a lithium carbon bond. Examples are methyllithium and n-butyllithium.

DCHA is dicyclohexylamine.

A counterion is the ion that accompanies an ionic species in order to maintain electric neutrality.

A tartrate is a salt or ester of the organic compound tartaric acid, a dicarboxylic acid.

The formula of the tartrate dianion is O⁻OC—CH(OH)—CH(OH)—COO⁻ or C₄H₄O₆²⁻.

A bisulfate is a salt of sulfuric acid, containing the HSO₄⁻ group.

In another embodiment of the invention, the base added to a compound of formula A is an alkyllithium, preferably n-butyllithium.

In an alternative embodiment, the base is added to a compound of formula A at low temperature, preferably below −70° C.

In an alternative embodiment, the compound of formula B is added at low temperature, preferably below −70° C.

In another embodiment, the method further comprises reducing the compound of Formula C

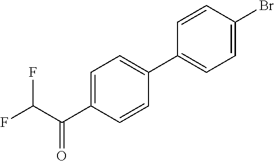 [SMILES: O=C(c1ccc(-c2ccc(Br)cc2)cc1)C(F)F]

to yield a compound of Formula D

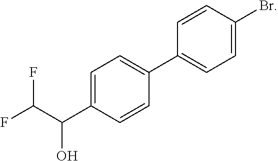 [SMILES: OC(c1ccc(-c2ccc(Br)cc2)cc1)C(F)F]

In another embodiment, the method further comprises reacting the compound of Formula D

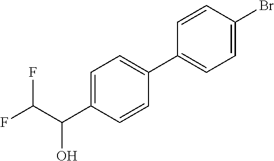 [SMILES: OC(c1ccc(-c2ccc(Br)cc2)cc1)C(F)F]

with one or more bases and a compound of Formula E

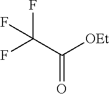

to yield a compound of Formula F

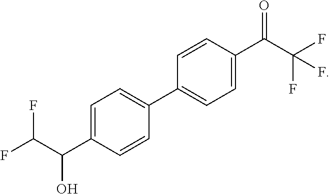 [SMILES: O=C(c1ccc(-c2ccc(C(O)C(F)F)cc2)cc1)C(F)(F)F]

In an alternative embodiment, the one or more bases added to the compound of Formula D comprise organolithium reagents, preferably methyllithium and n-butyllithium.

In an alternative embodiment, methyllithium is added to a compound of formula D at low temperature, preferably below −65° C.

In an alternative embodiment, following the addition of methyllithium to a compound of formula D, n-butyllithium is added at low temperature, preferably below −65° C.

In another embodiment, the method further comprises reacting the compound of Formula F

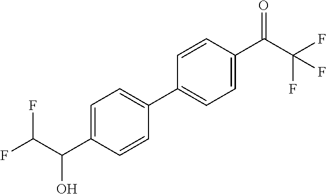 [SMILES: O=C(c1ccc(-c2ccc(C(O)C(F)F)cc2)cc1)C(F)(F)F]

with a compound of Formula G

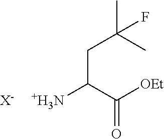

wherein X⁻ is a counterion;
in the presence of a reducing agent to yield a compound of Formula H

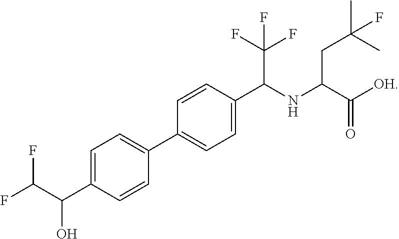 [SMILES: CC(C)(F)CC(NC(c1ccc(-c2ccc(C(O)C(F)F)cc2)cc1)C(F)(F)F)C(=O)O]

In an alternative embodiment, the compound of Formula G is

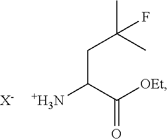

wherein X⁻ is bisulfate or tartrate, preferably tartrate.

In an alternative embodiment, the reducing agent is Zn(BH₄)₂.

In an alternative embodiment, the reduction is performed at low temperature, preferably not exceeding −5° C.

In another embodiment, the method further comprises reacting

the compound of Formula H

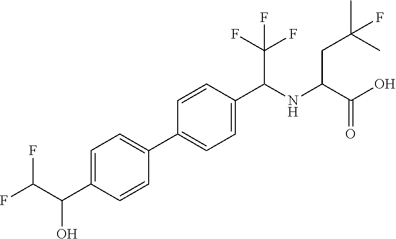 [SMILES: CC(C)(F)CC(NC(c1ccc(-c2ccc(C(O)C(F)F)cc2)cc1)C(F)(F)F)C(=O)O]

with a compound of Formula J

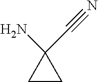

or a salt thereof in the presence of HATU to yield a compound of Formula I.

In an alternative embodiment, the reaction of a compound of formula H with a compound of formula J is performed in N,N-dimethylformamide.

In another embodiment, the invention is a method of preparing a compound of Formula I

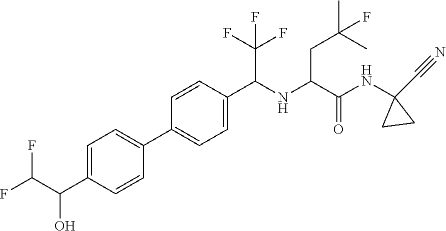 [SMILES: CC(C)(F)CC(NC(c1ccc(-c2ccc(C(O)C(F)F)cc2)cc1)C(F)(F)F)C(=O)NC1(C#N)CC1]

comprising
a) Reacting a compound of formula A

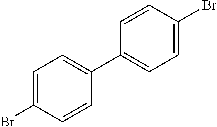 [SMILES: Brc1ccc(-c2ccc(Br)cc2)cc1]

with a base and a compound of Formula B

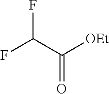

to yield of a compound of Formula C

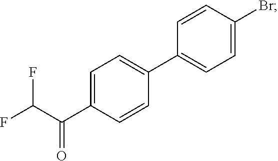 [SMILES: O=C(c1ccc(-c2ccc(Br)cc2)cc1)C(F)F]

b) Reducing the compound of Formula C to yield a compound of Formula D

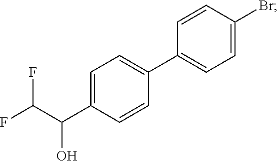 [SMILES: OC(c1ccc(-c2ccc(Br)cc2)cc1)C(F)F]

c. Reacting the compound of Formula D with one or more bases and a compound of Formula E

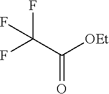

to yield a compound of Formula F

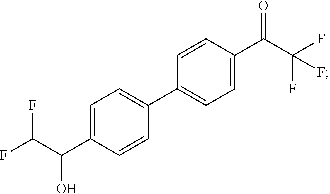 [SMILES: O=C(c1ccc(-c2ccc(C(O)C(F)F)cc2)cc1)C(F)(F)F]

d. Reacting the compound of Formula F with a compound of Formula G

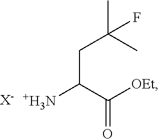

wherein X⁻ is bisulfate or tartrate, preferably tartrate,
in the presence of a reducing agent to yield a compound of Formula H

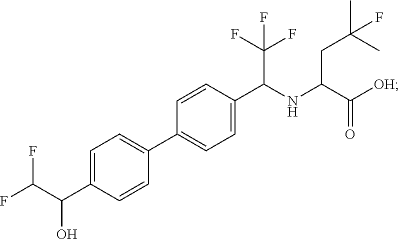 [SMILES: CC(C)(F)CC(NC(c1ccc(-c2ccc(C(O)C(F)F)cc2)cc1)C(F)(F)F)C(=O)O]

e. Reacting the compound of Formula H with a compound of Formula J

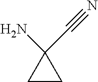

or a salt thereof
in the presence of O-(7-Azobenzotriazol-1-yl)-1,1,3,3-tetramethyluronium hexafluorophosphate (HATU) to yield a compound of Formula I.

In additional embodiments in step a, the base added to a compound of formula A is an alkyllithium, preferably n-butyllithium.

In additional embodiments, in step a, the base is added to a compound of formula A at low temperature, preferably below −70° C.

In additional embodiments, in step a, the compound of formula B is added at low temperature, preferably below −70° C.

In additional embodiments, in step c, the one or more bases added to the compound of Formula D comprise organolithium reagents, preferably methyllithium and n-butyllithium.

In additional embodiments, in step c, methyllithium is added to a compound of formula D at low temperature, preferably below −65° C.

In additional embodiments, in step c, following the addition of methyllithium to a compound of formula D, n-butyllithium is added at low temperature, preferably below −65° C.

In an additional embodiment, in step d, X⁻ is tartrate.

In an additional embodiment, in step d, the reducing agent is Zn(BH₄)₂.

In additional embodiments, in step d, the reduction is performed at low temperature, preferably not exceeding −5° C.

In an additional embodiment, in step e, the reaction of a compound of formula H with a compound of formula J is performed in N,N-dimethylformamide.

An alternative embodiment of the invention is a method of preparing a compound of Formula Ia

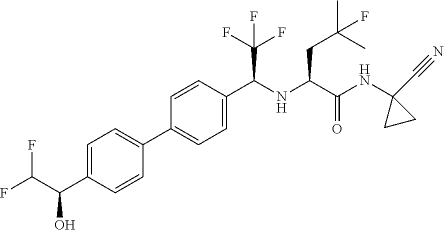 [SMILES: CC(C)(F)C[C@H](N[C@@H](c1ccc(-c2ccc([C@@H](O)C(F)F)cc2)cc1)C(F)(F)F)C(=O)NC1(C#N)CC1]

comprising
a) Reacting a compound of formula A

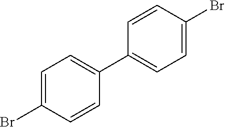 [SMILES: Brc1ccc(-c2ccc(Br)cc2)cc1]

with a base and a compound of formula B

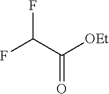

to yield of a compound of Formula C

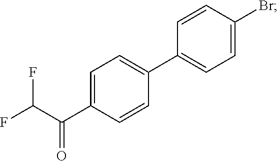 [SMILES: O=C(c1ccc(-c2ccc(Br)cc2)cc1)C(F)F]

b) Reducing the compound of Formula C to yield a compound of Formula Dd

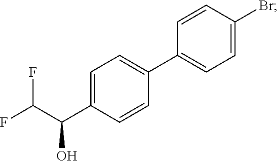 [SMILES: O[C@H](c1ccc(-c2ccc(Br)cc2)cc1)C(F)F]

c. Reacting the compound of Formula Dd with one or more bases and a compound of Formula E

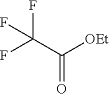

to yield a compound of Formula Ff

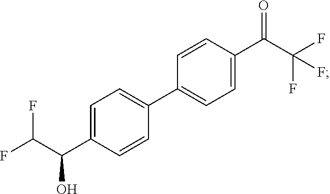 [SMILES: O=C(c1ccc(-c2ccc([C@@H](O)C(F)F)cc2)cc1)C(F)(F)F]

d. Reacting the compound of Formula Ff with a compound of Formula Gg

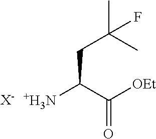

wherein X⁻ is bisulfate or tartrate, preferably tartrate,
in the presence of a reducing agent to yield a compound of Formula Hh

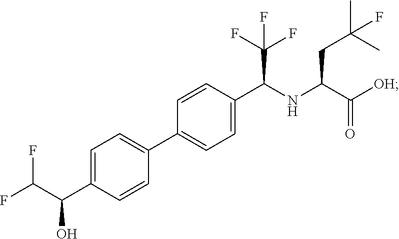 [SMILES: CC(C)(F)C[C@H](N[C@@H](c1ccc(-c2ccc([C@@H](O)C(F)F)cc2)cc1)C(F)(F)F)C(=O)O]

e. Reacting the compound of Formula Hh with a compound of Formula J

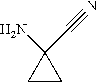

or a salt thereof
in the presence of a coupling reagent to yield a compound of Formula Ia.

In additional embodiments, in step a, the base added to a compound of Formula A is an alkyllithium, preferably n-butyllithium.

In additional embodiments, in step a, the base is added to a compound of Formula A at low temperature, preferably below −70° C.

In additional embodiments, in step a, the compound of Formula B is added at low temperature, preferably below −70° C.

In additional embodiments, in step b, the reduction is accomplished by the use of N,N-diethylaniline.borane complex and of a catalyst of Formula K

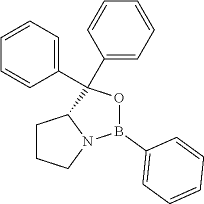 [SMILES: c1ccc(B2OC(c3ccccc3)(c3ccccc3)[C@H]3CCCN23)cc1]

In additional embodiments, in step b, the reduction is performed a low temperature, preferably below 0° C. and more preferably below −5° C.

In additional embodiments, in step c, the one or more bases added to the compound of Formula Dd comprise organolithium reagents, preferably methyllithium and n-butyllithium.

In additional embodiments, in step c, methyllithium is added to a compound of formula Dd at low temperature, preferably below −65° C.

In additional embodiments, in step c, following the addition of methyllithium to a compound of formula Dd, n-butyllithium is added at low temperature, preferably below −65° C.

In an additional embodiment, in step d, X⁻ is tartrate.

In an additional embodiment, in step d, the reducing agent is Zn(BH₄)₂.

In additional embodiments, in step d, the reduction is performed at low temperature, preferably not exceeding −5° C.

In additional embodiments, in step d, the compound of formula Hh may be isolated as a salt by the addition of a base, preferably a dialkylamine, most preferably diisopropylethylamine.

In an additional embodiment, in step e, the reaction of a compound of formula Hh with a compound of formula J is performed in N,N-dimethylformamide.

In an additional embodiment, the method further comprises isolating the compound of formula Ia from the reaction mixture by
i. the addition of phosphoric acid at a temperature above 25° C., preferably at 60° C., followed by
ii. the addition of water at a temperature above 25° C., preferably between 50 and 55° C. and then
iii. cooling down the mixture to 20 to 25° C.

In an alternative embodiment of the invention in the reaction of step a,
i. n-butyllithium was added while maintaining the temperature below −70° C.; and
ii. the compound of Formula B was also added while maintaining the temperature below −70° C.

In additional embodiments, the reduction of step c was accomplished by the use of a catalyst of Formula K

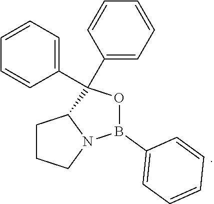 [SMILES: c1ccc(B2OC(c3ccccc3)(c3ccccc3)[C@H]3CCCN23)cc1]

In an alternative embodiment of the invention is a compound of Formula H

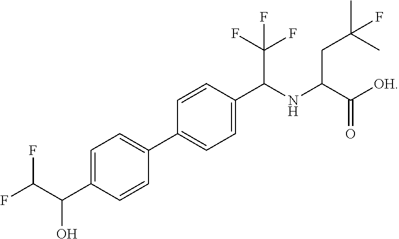 [SMILES: CC(C)(F)CC(NC(c1ccc(-c2ccc(C(O)C(F)F)cc2)cc1)C(F)(F)F)C(=O)O]

In an additional embodiment, the compound is of Formula Hh

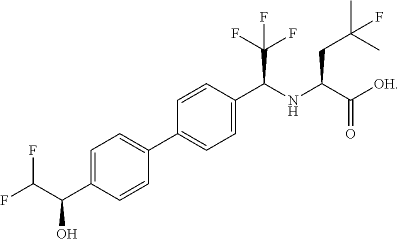 [SMILES: CC(C)(F)C[C@H](N[C@@H](c1ccc(-c2ccc([C@@H](O)C(F)F)cc2)cc1)C(F)(F)F)C(=O)O]

### Examples

### Step 1—1-(4′-Bromo[1,1′-biphenyl]-4-yl)-2,2-difluoro-ethanone synthesis

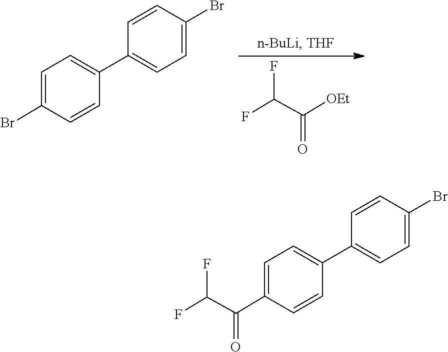 [SMILES: Brc1ccc(-c2ccc(Br)cc2)cc1 ; CCOC(=O)C(F)F ; O=C(c1ccc(-c2ccc(Br)cc2)cc1)C(F)F]

4,4′-Dibromo-1,1′-biphenyl (460 g, 1.48 mol) was dissolved in dry tetrahydrofuran (7.35 L) under inert atmosphere. The resulting solution was cooled to −76° C. and a 2.5 N solution of n-butyllithium in hexanes (610 mL, 1.53 mol) was added within 2.5 hours while keeping the internal temperature below −70° C. and ensuring a good dispersion of the n-butyllithium. After 15 min reaction time, ethyl 2,2-difluoroacetate (200 g, 1.61 mol) was added while keeping the internal temperature below −70° C. After 15 min reaction time, aqueous 3.0 N hydrochloric acid (750 mL) was added within 4 min and an increase in temperature to −45° C. was observed. The internal temperature was raised to 0° C., toluene was added (1.4 L) and the resulting mixture was vigorously stirred for 5 min. The mixture was settled for 10 min, the organic phase was separated and the aqueous phase was extracted with toluene (2×500 mL). Brine (750 mL) was added to the combined organic layers; the mixture was stirred for 5 min and was then allowed to settle for 30 min. The organic layer was collected and was concentrated under reduced pressure to a volume of about 1 L until an onset of crystallization was observed. As n-heptane (4 L) was added to the suspension, the temperature increased to 50° C. The suspension was then cooled to room temperature under gentle stirring. The obtained precipitate was filtered, rinsed with a 1 to 9 mixture of toluene and n-heptane (2×500 mL), and dried under vacuum at 40° C. to afford the desired product (308 g, 0.99 mol).

### Alternative 1-(4′-Bromo[1,1′-biphenyl]-4-yl)-2,2-difluoro-ethanone Synthesis

4,4′-Dibromo-1,1′-biphenyl (150 g, 0.48 mol) was dissolved in dry tetrahydrofuran (2 L) under inert atmosphere. The resulting solution was cooled to −72° C. and a 2.5 N solution of n-butyllithium in hexanes (194 mL, 0.48 mol) was added within 5.5 hours while keeping the internal temperature below −70° C. and ensuring a good dispersion of the n-butyllithium. After 5 min reaction time, ethyl 2,2-difluoroacetate (65.7 g, 0.53 mol) was added within 30 min while the internal temperature was raised to −60° C. After 30 min reaction time, aqueous 10% ammonium chloride (750 mL) was added within 30 min and the temperature was raised to −40° C. The internal temperature was raised to −5° C., methyl tert-butylether was added (750 mL) and the resulting mixture was vigorously stirred for 10 min. The mixture was warmed to 20° C. and stirred for 20 min and was allowed to settle for 30 min. The aqueous layer was discarded and the organic phase was washed with aqueous 5% sodium sulfate (4×750 mL). The organic phase was concentrated under reduced pressure to a volume of about 600 mL while the internal temperature was increased to 40° C. 2-Propanol (520 mL) was added dropwise while the total volume was kept at about 700 mL and the internal temperature was maintained at 40° C. A seed of the desired product (0.75 g, 2.4 mmol) was added to the mixture which was stirred at 40° C. for 16 hours. In order to induce even more precipitation, 2-propanol (1.5 L) was added while the total volume was kept at about 900 mL and the internal temperature was maintained at 40° C. After 12 hours stirring at 40° C., the suspension was cooled to 18° C. and was stirred at this temperature for 24 hours. The obtained precipitate was filtered, rinsed with 2-propanol (200 mL), and dried under vacuum at 40° C. to afford the desired product (107 g, 0.34 mol).

### Step 2-4′-Bromo-α-(difluoromethyl)-(αR)-[1,1′-biphenyl]-4-methanol Synthesis

### Step 2a—Preparation of the (R)-(+)-Ph-OAB Catalyst

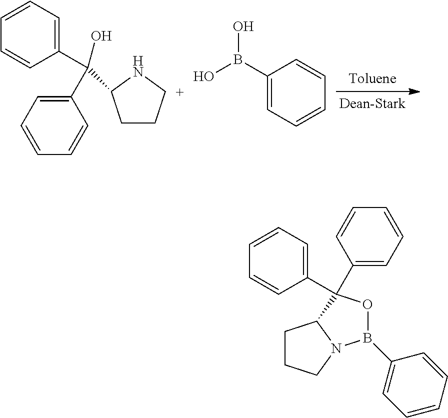

(R)-(+)-Diphenyl(pyrrolidin-2-yl)methanol (25.6 g, 99 mmol) and phenylboronic acid (12.94 g, 104 mmol) were dissolved in toluene (1 L) under inert atmosphere. The resulting mixture was refluxed for 20 hours while the generated water was azeotropically removed using a Dean-Stark apparatus. Complete conversion of (R)-(+)-diphenyl(pyrrolidin-2-yl)methanol was ensured by NMR and the obtained yellow solution was kept under inert atmosphere at room temperature until being engaged as such in the next step.

### Step 2b—Asymmetric reduction of 1-(4′-Bromo[1,1′-biphenyl]-4-yl)-2,2-difluoro-ethanone

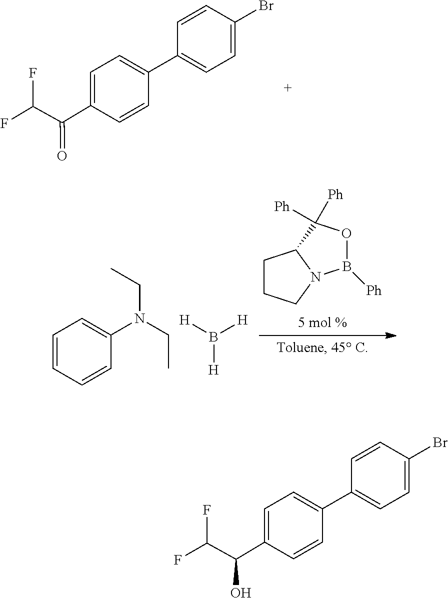

A solution of (R)-(+)-Ph-OAB in toluene obtained in the previous step (497 mL, 48.2 mmol) was added to a solution of N,N-diethylaniline.borane complex (189 mL, 1.06 mol) in toluene (1.2 L) and the resulting mixture was heated to 45° C. A solution of 1-(4′-bromo-[1,1′-biphenyl]-4-yl)-2,2-difluoroethanone (300 g, 0.96 mol) in toluene (1.5 L) was added dropwise over 3 hours. After completion of the addition, the reaction mixture was stirred for 1 hour at 45° C. Aqueous 3.0 N hydrochloric acid (1.2 L) was added very slowly while keeping the temperature below 55° C. After completion of the addition, the mixture was stirred for 1 hour at 45° C. The reaction mixture was cooled to room temperature. The aqueous phase of the reaction mixture was discarded while the organic phase was washed with brine (600 mL) and concentrated under reduced pressure. The crude product was taken up in a 2 to 3 mixture of methanol and water (2.85 L) and was gently stirred at 40° C. for 18 hours. The obtained solid was filtered, rinsed with a 2 to 3 mixture of methanol and water (2×500 mL) and was dried under vacuum at 40° C. to afford the desired product (297 g, 0.95 mol).

### Step 2c—Recrystallization of 4′-Bromo-α-(difluoromethyl)-(αR)-[1,1′-biphenyl]-4-methanol

4′-Bromo-α-(difluoromethyl)-(αR)-[1,1′-biphenyl]-4-methanol (546 g) was added to dry toluene (1.09 L), the temperature was raised to 65° C. and heptane (1.64 L) was slowly added over 45 min while maintaining the temperature at 65° C. Heating was stopped and the mixture was allowed to cool to room temperature overnight while being gently stirred. Precipitation began as the mixture cooled to about 55° C. Stirring was stopped and the solids were allowed to settle. After filtration, the obtained cake was slurried with a 2 to 3 mixture of toluene and heptane (2.5 L) and was then rinsed with n-heptane (4 L). The solids were then dried under vacuum to afford pure 4′-bromo-α-(difluoromethyl)-(αR)-[1,1′-biphenyl]-4-methanol.

### Step 3—1-[4′-[(1R)-2,2-Difluoro-1-hydroxyethyl][1,1′-biphenyl]-4-yl]-2,2,2-trifluoroethanone Synthesis

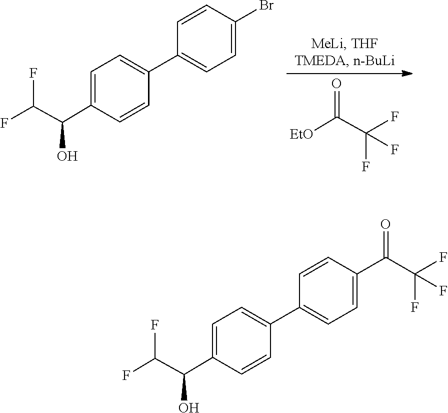 [SMILES: O[C@H](c1ccc(-c2ccc(Br)cc2)cc1)C(F)F ; CCOC(=O)C(F)(F)F ; O=C(c1ccc(-c2ccc([C@@H](O)C(F)F)cc2)cc1)C(F)(F)F]

(R)-1-(4′-Bromo-[1,1′-biphenyl]-4-yl)-2,2-difluoroethanol (720 g, 2.30 mol) was dissolved in dry tetrahydrofuran (3.6 L) under inert atmosphere and the resulting solution was cooled to −70° C. A 1.6 M solution of methyllithium in diethylether (1.58 L, 2.53 mol) was added over 1 hour while keeping the temperature between −70 and −65° C. After 5 min reaction time, N,N,N′,N′-tetramethylethane-1,2-diamine (1.15 L, 7.59 mol) was added within 15 min while an increase in the temperature from −70 to −65° C. was observed. The resulting mixture was stirred for 5 min to let the temperature cool again to −70° C. and then a 2.5 M solution of n-butyllithium in n-hexan (1.012 L, 2.53 mol) was added and an increase in the temperature from −70 to −65° C. was observed. After 10 min reaction time, ethyl 2,2,2-trifluoroacetate (360 mL, 3.03 mol) was added dropwise within 20 min and an increase in the temperature to −50° C. was observed. The reaction mixture was stirred at −50° C. for 30 min and aqueous 6 N hydrochloric acid (3.83 L) was added within 10 min while the temperature is allowed to increase from −50 to 0° C. Methyl tert-butylether (4 L) was added and the resulting mixture was stirred for 10 min. The aqueous layer was separated; the organic layer was washed with brine (2.5 L) and concentrated under reduced pressure. A 2 to 3 mixture of methyl tert-butylether and petroleum ether (800 mL) was added to the crude product and the resulting solution was purified by silica gel column chromatography using a gradient of petroleum ether in methyl tert-butylether. After collection of the fractions of interest and concentration under reduced pressure, the desired product was isolated as solid (728 g, 2.20 mol).

### Step 4—(2S)-2-[[(1S)-1-[4-[4-[(1R)-2,2-difluoro-1-hydroxy-ethyl]phenyl]phenyl]-2,2,2-trifluoro-ethyl]amino]-4-fluoro-4-methyl-pentanoic acid Synthesis

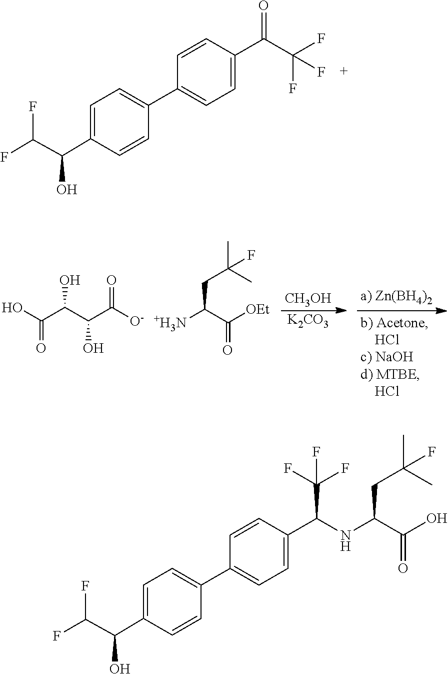

A reactor was charged with (S)-γ-fluoroleucine ethyl ester tartrate (688 g, 2.1 mol), potassium carbonate (1.22 kg, 8.74 mol) and methanol (1.5 L). After addition of a solution of (R)-1-(4′-(2,2-difluoro-1-hydroxyethyl)-[1,1′-biphenyl]-4-yl)-2,2,2-trifluoroethanone (596 g, 1.80 mol) in methanol (1.9 L), water (31.5 mL, 1.75 mmol) was added while a slight exotherm was observed. The resulting mixture was warmed to 50° C. internal temperature and was stirred at this temperature for 9 hours. The temperature was lowered to 10° C. and the reaction mixture was stirred further at this temperature for 18 hours. A second reactor was charged with zinc(II) chloride (484 g, 3.55 mol) and 1,2-dimethoxyethane (2.6 L) and the resulting mixture was cooled to −20° C. Sodium tetrahydroborate (270 g, 7.14 mol) was added in two portions while an increase in temperature from −20 to −6° C. was observed. The reaction mixture was cooled to −15° C. and was stirred at this temperature for 1 hour. Acetonitrile (3.95 L) was added, the temperature was adjusted to −15 to −20° C. and the mixture was aged for 30 min. The temperature of the second reactor was lowered between −50 and −45° C. and the content of the first reactor pre-cooled at −5° C. was added over 30 min while keeping the temperature below −5° C. The first reactor was rinsed with methanol (500 mL) which was added to the content of the second reactor. The temperature was adjusted to −20 to −10° C. and the reaction mixture was aged for 90 min. Acetone (2.25 L) was added over 15 min, allowing the temperature to reach −5° C. After aging 30 min, the reaction mixture was cooled to −20° C. and aqueous 3 N hydrochloric acid (5.8 L) was added within 30 min, keeping the temperature below 0° C. After addition of methyl tert-butyl ether (8.4 L) and water (1 L), the resulting heterogeneous mixture was stirred for 18 hours. Additional water (1.6 L) was added and the resulting suspension was filtered. The filter cake was washed with methyl tert-butylether (2 L) which was added to the filtrate. After separation of the aqueous phase, heptane (400 mL) was added to the organic layer which was cooled to 10° C. before being extracted with aqueous 3 N sodium hydroxide (1.7 L) followed by aqueous 1 N sodium hydroxide (2×2.5 L). After addition of methyl tert-butylether (7.5 L) to the combined aqueous layers, the temperature was lowered to 10° C. and aqueous 6 N hydrochloric acid (2.1 L) was added while keeping the temperature below 15° C. After separation from the aqueous phase, the organic phase was washed with water (5×2.5 L) and concentrated to a volume of about 5 L under reduced pressure. The concentrated organic phase was heated to 30° C. and n-heptane (7.4 L) was slowly added under stirring. The obtained thick suspension was cooled to 5° C., was filtered and the filter cake was washed with a mixture 4 to 1 mixture of n-heptane and methyl tert-butylether (2.6 L) before being dried at 40° C. under vacuum to afford the desired product (647 g, 1.40 mol).

In an additional experiment, the above synthesis was conducted with the bisulfate salt of the (S)-γ-fluoroleucine ethyl ester. Both tartrate and bisulfate esters were prepared as described in WO2013148554. In yet another experiment, the diisopropylammonium salt of the final product was isolated which allowed for a slight increase in optical purity of the salt versus the free acid.

### Step 5—(2S)-N-(1-cyanocyclopropyl)-2-[[(1S)-1-[4-[4-[(1R)-2,2-difluoro-1-hydroxy-ethyl]phenyl]phenyl]-2,2,2-trifluoro-ethyl]amino]-4-fluoro-4-methyl-pentanamide Synthesis

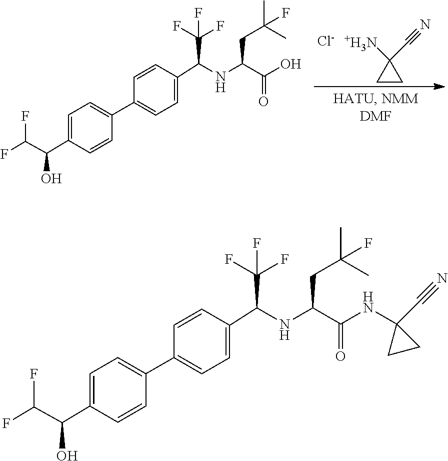 [SMILES: CC(C)(F)C[C@H](N[C@@H](c1ccc(-c2ccc([C@@H](O)C(F)F)cc2)cc1)C(F)(F)F)C(=O)O ; [Cl-] ; N#CC1([NH3+])CC1 ; CC(C)(F)C[C@H](N[C@@H](c1ccc(-c2ccc([C@@H](O)C(F)F)cc2)cc1)C(F)(F)F)C(=O)NC1(C#N)CC1]

(2S)-2-[[(1S)-1-[4-[4-[(1R)-2,2-difluoro-1-hydroxy-ethyl]phenyl]phenyl]-2,2,2-trifluoro-ethyl]amino]-4-fluoro-4-methyl-pentanoic acid (545 g, 1.18 mol) and 1-aminocyclopropanecarbonitrile hydrochloride (167 g, 1.41 mol) are dissolved in N,N-dimethylformamide (5 L) and the resulting solution was cooled to 0° C. 4-Methylmorpholine (327 mL, 2.94 mol) was added within 20 min while keeping the temperature below 2° C. To the obtained suspension, O-(7-azobenzotriazol-1-yl)-1,1,3,3-tetramethyluronium hexafluorophosphate (547 g, 1.41 mol) was added in two portions while an increase in temperature from 0 to 5° C. was observed. After 7 hours reaction time at 5° C., the reaction mixture was allowed to reach room temperature and was stirred further for 18 hours. The temperature of the reaction mixture was increased to 60° C. over 90 min and aqueous 4% phosphoric acid (6.52 L) was added. After completion of the addition, a turbid mixture was obtained. Water (8.75 L) was added within 90 min at a temperature between 50 and 55° C. and the resulting mixture was stirred at this temperature for 2 hours. The reaction mixture was then allowed to cool to 20 to 25° C. over 18 hours. The obtained suspension was filtered, the reactor was washed with water (800 mL) which was used to rinse the cake. The cake was sequentially slurry washed with a 1 to 3 mixture of N,N-dimethylformamide and water (1.5 L) and then with water (3×3 L) before being dried by applying a nitrogen flow to afford the desired product as white solid (610 g, 1.16 mol).

The following numbered examples are embodiments of the invention:

1. A method of preparing a compound of Formula I

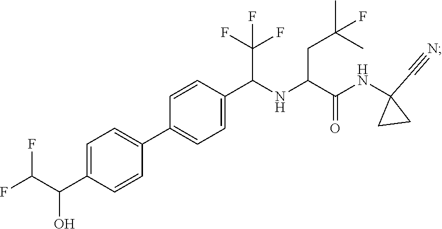 [SMILES: CC(C)(F)CC(NC(c1ccc(-c2ccc(C(O)C(F)F)cc2)cc1)C(F)(F)F)C(=O)NC1(C#N)CC1]

comprising
reacting a compound of Formula A

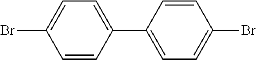 [SMILES: Brc1ccc(-c2ccc(Br)cc2)cc1]

with a base and a compound of Formula B

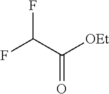

to yield a compound of Formula C

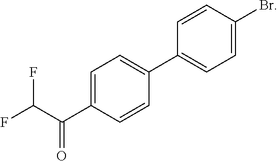 [SMILES: O=C(c1ccc(-c2ccc(Br)cc2)cc1)C(F)F]

2. The method of claim 1, wherein the base is an alkyllithium, preferably n-butyllithium.

3. The method of anyone of claims 1-2, wherein the base is added to a compound of formula A at low temperature, preferably below −70° C.

4. The method of anyone of claims 1-3, wherein the compound of formula B is added at low temperature, preferably below −70° C.

5. The method of

anyone of claims 1-4, further comprising reducing the compound of Formula C

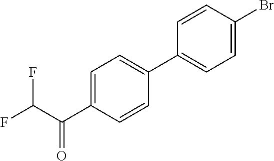 [SMILES: O=C(c1ccc(-c2ccc(Br)cc2)cc1)C(F)F]

to yield a compound of Formula D

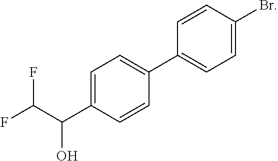 [SMILES: OC(c1ccc(-c2ccc(Br)cc2)cc1)C(F)F]

6. The method of

claim 5, further comprising reacting the compound of Formula D

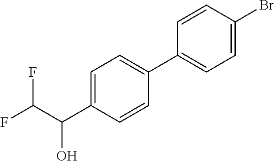 [SMILES: OC(c1ccc(-c2ccc(Br)cc2)cc1)C(F)F]

with one or more bases and a compound of Formula E

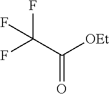

to yield a compound of Formula F

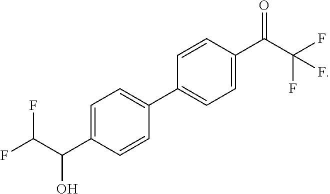 [SMILES: O=C(c1ccc(-c2ccc(C(O)C(F)F)cc2)cc1)C(F)(F)F]

7. The method of claim 6, wherein the one or more bases comprise organolithium reagents, preferably methyllithium and n-butyllithium.

8. The method of anyone of claims 6-7, wherein methyllithium is added to a compound of formula D at low temperature, preferably below −65° C.

9. The method of claim 8, wherein following the addition of methyllithium to a compound of formula D, n-butyllithium is added at low temperature, preferably below −65° C.

10. The method of

anyone of claims 6-9, further comprising reacting the compound of Formula F

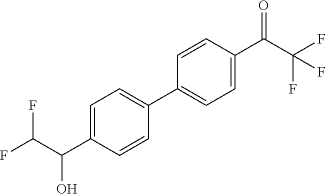 [SMILES: O=C(c1ccc(-c2ccc(C(O)C(F)F)cc2)cc1)C(F)(F)F]

with a compound of Formula G

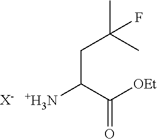

wherein X⁻ is a counterion;
in the presence of a reducing agent to yield a compound of Formula H

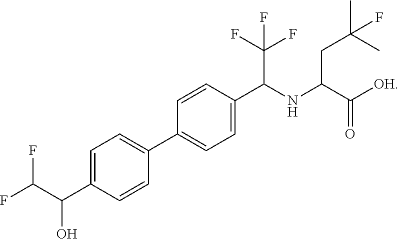 [SMILES: CC(C)(F)CC(NC(c1ccc(-c2ccc(C(O)C(F)F)cc2)cc1)C(F)(F)F)C(=O)O]

11. The method of claim 10 wherein the compound of Formula G is

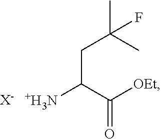

wherein X⁻ is bisulfate or tartrate, preferably tartrate.

12. The method of anyone of claims 10-11, wherein the reducing agent is Zn(BH₄)₂.

13. The method of anyone of claims 10-12, wherein the reduction is performed at low temperature, preferably not exceeding −5° C.

14. The method of

anyone of claims 10-13, further comprising reacting
the compound of Formula H

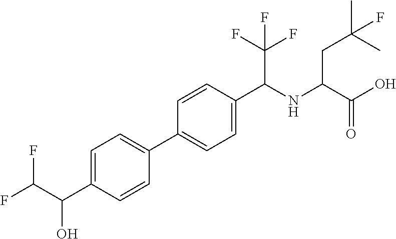 [SMILES: CC(C)(F)CC(NC(c1ccc(-c2ccc(C(O)C(F)F)cc2)cc1)C(F)(F)F)C(=O)O]

with a compound of Formula J

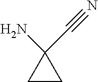

or a salt thereof in the presence of HATU to yield a compound of Formula I.

15. The method of claim 14, wherein in the reaction of a compound of formula H with a compound of formula J is performed in N,N-dimethylformamide.

16. A method of preparing a compound of Formula I

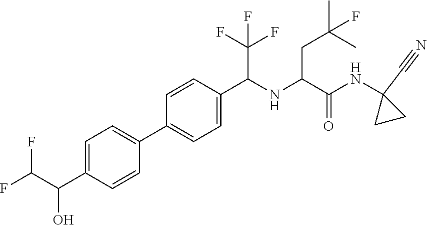 [SMILES: CC(C)(F)CC(NC(c1ccc(-c2ccc(C(O)C(F)F)cc2)cc1)C(F)(F)F)C(=O)NC1(C#N)CC1]

comprising,
a) Reacting a compound of Formula A

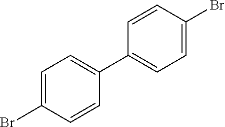 [SMILES: Brc1ccc(-c2ccc(Br)cc2)cc1]

with a base and a compound of Formula B

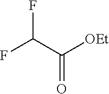

to yield of a compound of Formula C

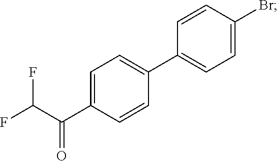 [SMILES: O=C(c1ccc(-c2ccc(Br)cc2)cc1)C(F)F]

b) Reducing the compound of Formula C to yield a compound of Formula D

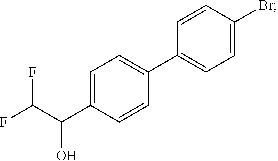 [SMILES: OC(c1ccc(-c2ccc(Br)cc2)cc1)C(F)F]

c. Reacting the compound of Formula D with one or more bases and a compound of Formula E

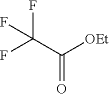

to yield a compound of Formula F

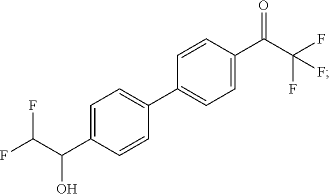 [SMILES: O=C(c1ccc(-c2ccc(C(O)C(F)F)cc2)cc1)C(F)(F)F]

d. Reacting the compound of Formula F with a compound of Formula G

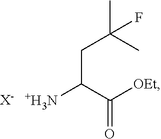

wherein X⁻ is bisulfate or tartrate, preferably tartrate,
in the presence of a reducing agent to yield a compound of Formula H

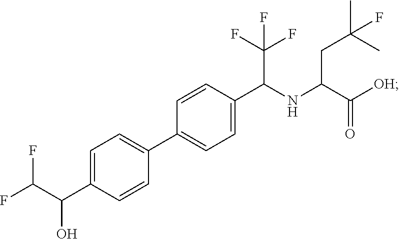 [SMILES: CC(C)(F)CC(NC(c1ccc(-c2ccc(C(O)C(F)F)cc2)cc1)C(F)(F)F)C(=O)O]

e. Reacting the compound of Formula H with a compound of Formula J

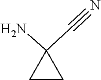

or a salt thereof
in the presence of O-(7-Azobenzotriazol-1-yl)-1,1,3,3-tetramethyluronium hexafluorophosphate (HATU) to yield a compound of Formula I.

17. The method of claim 16, wherein in step a, the base is an alkyllithium, preferably n-butyllithium.

18. The method of anyone of claims 16-17, wherein in step a, the base is added to a compound of formula A at low temperature, preferably below −70° C.

19. The method of anyone of claims 16-18, wherein in step a, the compound of formula B is added at low temperature, preferably below −70° C.

20. The method of anyone of claims 16-19, wherein in step c, the one or more bases comprise organolithium reagents, preferably methyllithium and n-butyllithium.

21. The method of claim 20, wherein in step c, methyllithium is added to a compound of formula D at low temperature, preferably below −65° C.

22. The method of claims 20-or 21, wherein in step c, following the addition of methyllithium to a compound of formula D, n-butyllithium is added at low temperature, preferably below −65° C.

23. The method of anyone of claims 16-22, wherein in step d, X⁻ is tartrate.

24. The method of anyone of claims 16-23, wherein in step d, the reducing agent is Zn(BH₄)₂.

25. The method of anyone of claims 16-24, wherein in step d, the reduction is performed at a temperature not exceeding 0° C., preferably not exceeding −5° C.

26. The method of anyone of claims 16-25, wherein in step e, the reaction of a compound of formula H with a compound of formula J is performed in N,N-dimethylformamide.

27. A method of preparing a compound of Formula Ia

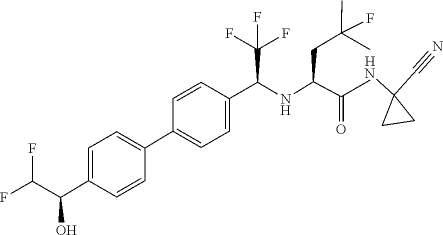

comprising
a) Reacting a compound of formula A

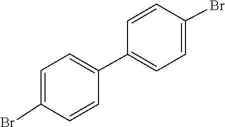 [SMILES: Brc1ccc(-c2ccc(Br)cc2)cc1]

with a base and a compound of formula B

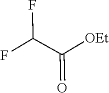

to yield of a compound of Formula C

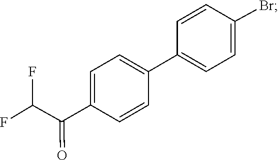 [SMILES: O=C(c1ccc(-c2ccc(Br)cc2)cc1)C(F)F]

b) Reducing the compound of Formula C to yield a compound of Formula Dd

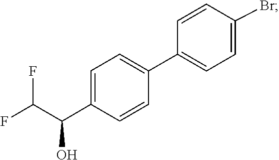 [SMILES: O[C@H](c1ccc(-c2ccc(Br)cc2)cc1)C(F)F]

c. Reacting the compound of Formula Dd with one or more bases and a compound of Formula E

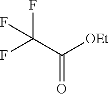

to yield a compound of Formula Ff

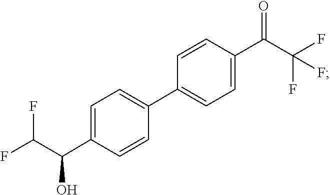 [SMILES: O=C(c1ccc(-c2ccc([C@@H](O)C(F)F)cc2)cc1)C(F)(F)F]

d. Reacting the compound of Formula Ff with a compound of Formula Gg

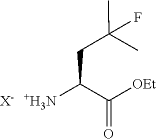

wherein X⁻ is bisulfate or tartrate, preferably tartrate,
in the presence of a reducing agent to yield a compound of Formula Hh

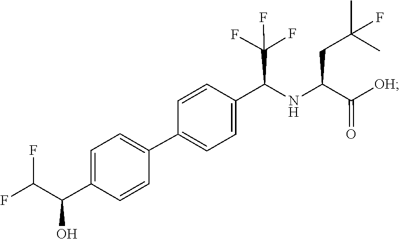 [SMILES: CC(C)(F)C[C@H](N[C@@H](c1ccc(-c2ccc([C@@H](O)C(F)F)cc2)cc1)C(F)(F)F)C(=O)O]

e. Reacting the compound of Formula Hh with a compound of Formula J

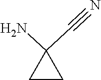

or a salt thereof
in the presence of a coupling reagent to yield a compound of Formula Ia.

28. The method of claim 27, wherein in step a, the base is an alkyllithium, preferably n-butyllithium.

29. The method of anyone of claims 27-28, wherein in step a, the base is added to a compound of formula A at low temperature, preferably below −70° C.

30. The method of anyone of claims 27-29, wherein in step a, the compound of formula B is added at low temperature, preferably below −70° C.

31. The method of anyone of claims 27-30, wherein in step b, the reduction is preferably accomplished by the use of N,N-diethylaniline.borane complex and of a catalyst of Formula K

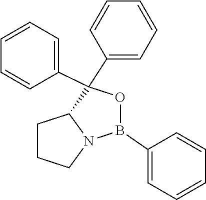 [SMILES: c1ccc(B2OC(c3ccccc3)(c3ccccc3)[C@H]3CCCN23)cc1]

32. The method of anyone of claims 27-31, wherein in step b, the reduction is performed a temperature below 0° C. and more preferably below −5° C.

33. The method of anyone of claims 27-32, wherein in step c, the one or more bases comprise organolithium reagents, preferably methyllithium and n-butyllithium.

34. The method of claim 33, wherein in step c, methyllithium is added to a compound of formula Dd at low temperature, preferably below −65° C.

35. The method of claim 33 or 34, wherein in step c, following the addition of methyllithium to a compound of formula Dd, n-butyllithium is added at low temperature, preferably below −65° C.

36. The method of anyone of claims 27-35, wherein in step d, X⁻ is tartrate.

37. The method of anyone of claims 27-36, wherein in step d, the reducing agent is Zn(BH₄)₂.

38. The method of anyone of claims 27-37, wherein in step d, the reduction is performed at a temperature not exceeding 0° C., preferably not exceeding −5° C.

39. The method of anyone of claims 27-38, wherein in step d, the compound of formula Hh may be isolated as a salt by the addition of a base, preferably a dialkylamine, more preferably diisopropylethylamine.

40. The method of anyone of claims 27-39, wherein in step e, the reaction of a compound of formula Hh with a compound of formula J is performed in N,N-dimethylformamide.

41. The method of anyone of claims 27-40, further comprising isolating the compound of formula Ia from the reaction mixture by
i. the addition of phosphoric acid at a temperature above 25° C., preferably at 60° C., followed by
ii. the addition of water at a temperature above 25° C., preferably between 50 and 55° C. and then
iii. cooling down the mixture to 20 to 25° C.

42. The method of any one of claims 16-41, wherein in the reaction of step a,
i. n-butyllithium was added while maintaining the temperature below −70° C.; and
ii. the compound of Formula B was also added while maintaining the temperature below −70° C.

43. The method of any one of claims 16-42, wherein the reduction of step c was accomplished by the use of a catalyst of Formula K

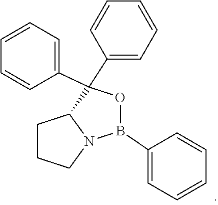 [SMILES: c1ccc(B2OC(c3ccccc3)(c3ccccc3)[C@H]3CCCN23)cc1]

44. A compound of Formula H

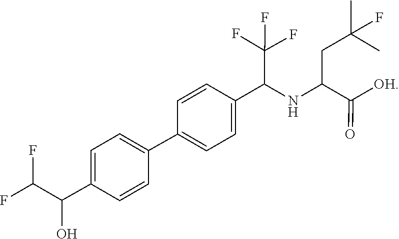 [SMILES: CC(C)(F)CC(NC(c1ccc(-c2ccc(C(O)C(F)F)cc2)cc1)C(F)(F)F)C(=O)O]

45. The compound of claim 44, wherein the compound is of Formula Hh

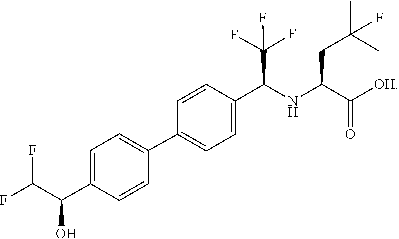 [SMILES: CC(C)(F)C[C@H](N[C@@H](c1ccc(-c2ccc([C@@H](O)C(F)F)cc2)cc1)C(F)(F)F)C(=O)O]

1. A method of preparing a compound of Formula I 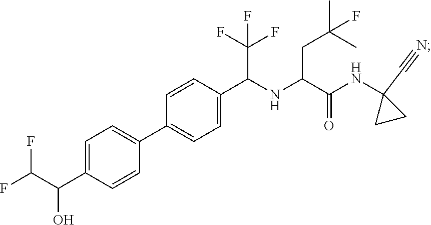 [SMILES: CC(C)(F)CC(NC(c1ccc(-c2ccc(C(O)C(F)F)cc2)cc1)C(F)(F)F)C(=O)NC1(C#N)CC1]
comprising
reacting a compound of Formula A 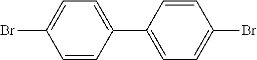 [SMILES: Brc1ccc(-c2ccc(Br)cc2)cc1]
with a base and a compound of Formula B 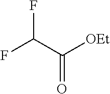
to yield a compound of Formula C

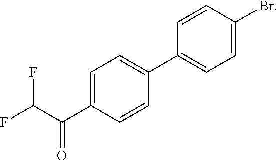 [SMILES: O=C(c1ccc(-c2ccc(Br)cc2)cc1)C(F)F]

2. The method of claim 1, wherein the base is an alkyllithium.

3. The method of claim 1, wherein the base is added to a compound of formula A at low temperature.

4. The method of claim 1, wherein the compound of formula B is added at low temperature.

5. The method of claim 1, further comprising reducing the compound of Formula C 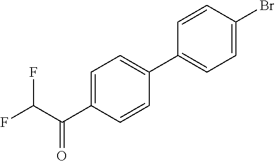 [SMILES: O=C(c1ccc(-c2ccc(Br)cc2)cc1)C(F)F]
to yield a compound of Formula D

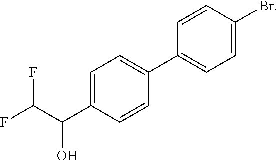 [SMILES: OC(c1ccc(-c2ccc(Br)cc2)cc1)C(F)F]

6. The method of claim 5, further comprising reacting the compound of Formula D 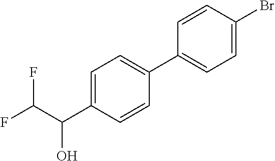 [SMILES: OC(c1ccc(-c2ccc(Br)cc2)cc1)C(F)F]
with one or more bases and a compound of Formula E 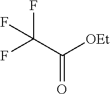
to yield a compound of Formula F

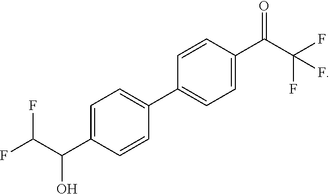 [SMILES: O=C(c1ccc(-c2ccc(C(O)C(F)F)cc2)cc1)C(F)(F)F]

7. The method of claim 6, wherein the one or more bases comprise organolithium reagents.

8. The method of claim 6, wherein methyllithium is added to a compound of formula D at low temperature.

9. The method of claim 8, wherein following the addition of methyllithium to a compound of formula D, n-butyllithium is added at low temperature.

10. The method of claim 6, further comprising reacting the compound of Formula F 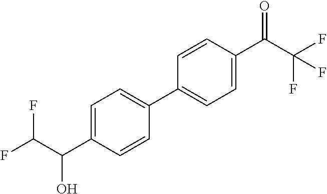 [SMILES: O=C(c1ccc(-c2ccc(C(O)C(F)F)cc2)cc1)C(F)(F)F]
with a compound of Formula G 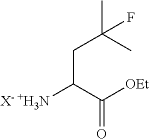
wherein X⁻ is a counterion;
in the presence of a reducing agent to yield a compound of Formula H

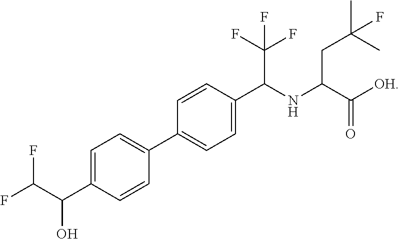 [SMILES: CC(C)(F)CC(NC(c1ccc(-c2ccc(C(O)C(F)F)cc2)cc1)C(F)(F)F)C(=O)O]

11. The method of claim 10 wherein the compound of Formula G is 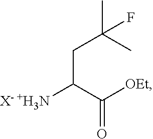
wherein X⁻ is bisulfate or tartrate.

12. The method of claim 10, wherein the reducing agent is Zn(BH₄)₂.

13. The method of claim 10, wherein the reduction is performed at low temperature.

14. The method of claim 10, further comprising reacting the compound of Formula H 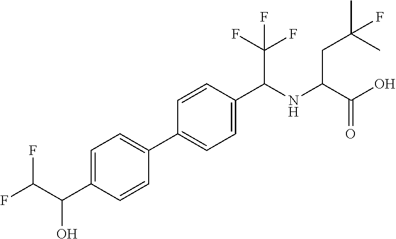 [SMILES: CC(C)(F)CC(NC(c1ccc(-c2ccc(C(O)C(F)F)cc2)cc1)C(F)(F)F)C(=O)O]
with a compound of Formula J 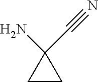
or a salt thereof in the presence of HATU to yield a compound of Formula I.

15. The method of claim 14, wherein in the reaction of a compound of formula H with a compound of formula J is performed in N,N-dimethylformamide.

16. The method of claim 1, wherein the base is n-butyllithium.

17. The method of claim 3, wherein the base is added to a compound of formula A at a temperature below −70° C.

18. The method of claim 4, wherein the compound of formula B is added at a temperature below −70° C.

19. The method of claim 7, wherein the organolithium reagents are methyllithium and n-butyllithium.

20. The method of claim 8, wherein methyllithium is added to a compound of formula D at a temperature below −65° C.

21. The method of claim 9, wherein following the addition of methyllithium to a compound of formula D, n-butyllithium is added at a temperature-below −65° C.

22. The method of claim 11, wherein X⁻ is tartrate.

23. The method of claim 13, wherein the reduction is performed at a temperature not exceeding −5° C.
